(19) United States
(12) Patent Application Publication
(10) Pub. No.: US 2008/0124295 A1
(43) Pub. Date: May 29, 2008

(54) Process for elimination of the odor retained by keratinic materials in contact with water treated with a halogenated disinfectant

(76) Inventors: Albert Duranton, Maison Laffite (FR); Francis Pruche, Senlis (FR); Stephane Lacoutiere, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/898,248

(22) Filed: Sep. 11, 2007

Related U.S. Application Data
(60) Provisional application No. 60/845,502, filed on Sep. 19, 2006.

(30) Foreign Application Priority Data
Sep. 12, 2006 (FR) 0653690

(51) Int. Cl. A61K 8/46; A61K 8/84; A61Q 5/12; A61Q 5/00

(52) U.S. Cl. 424/70.15; 424/70.1; 424/70.11

(57) ABSTRACT

The present disclosure relates to a cosmetic process to reduce or eliminate the odor retained by keratinic materials placed in contact with water treated with a halogenated disinfectant, such as a chlorinated disinfectant and/or a brominated disinfectant, comprising applying onto the keratinic materials at least one composition comprising at least one aminoalkane sulphonic or sulphonothioic acid compound or a salt thereof or an amide derivative thereof in a cosmetically acceptable medium, then if necessary rinsing with water.

This application claims benefit of U.S. Provisional Application No. 60/845,502, filed Sep. 19, 2006, the contents of which are incorporated herein by reference. This application also claims benefit of priority under 35 U.S.C. § 119 to French Patent Application FR 06/53690, filed Sep. 12, 2006, the contents of which are also incorporated herein by reference.

Disclosed herein is a cosmetic process for reducing or eliminating the odor retained by keratinic materials placed in contact with water treated with a halogenated disinfectant, the process comprising applying onto the keratinic materials at least one composition containing, in a cosmetically acceptable medium, at least one aminoalkane sulfonic or sulfono-thioic acid compound or a salt thereof or an amide derivative thereof, then performing a water rinsing if necessary.

Swimming pools are generally treated with halogenated disinfectant agents, for example, chlorinated disinfectants and/or brominated disinfectants which may cause an unpleasant smell on keratinic materials such as the skin and the hair, perceptible even after several days and even after repeated washings with a soap, a shower gel or a shampoo on leaving the swimming pool.

The disinfectant most commonly used in swimming pools is chlorine. When a chlorinated disinfectant (hydrochloric acid and sodium hypochlorite) is added to water, it generally takes the active form of a hypochlorous acid. Hypochlorous acid is measured in water as available free chlorine.

UV radiation, a low pH and splashing may all contribute to the decrease in the level of free chlorine: chlorine reacts with ammonia and nitrogen deriving from pollutant substances produced by swimmers such as perspiration, urine and sun oils to produce combined chloramines.

Chloramines combined with pollutant substances are malodorous and are less effective than free chlorine. They can be irritating to the body and the eyes when they are present in excess. The odor is an indication that the free chlorine has been exhausted and is now in the form of combined chloramines.

It would be desirable, therefore, to find a process which makes it possible to efficiently decrease or suppress the odor retained by keratinic materials when they are in contact with water treated with a halogenated disinfectant compound, such as a chlorinated disinfectant and/or a brominated disinfectant.

Certain aminoalkane sulfonic, sulfinic or sulfonothioic acid compounds such as taurine or homotaurine are known for their applications in cosmetics and, for instance, have been used for many years in the skin and/or hair cleansing field. They are also known for hair care, for example, for the stimulation of hair growth in French Patent No. FR-2 668 928 or skin care, such as for the treatment of dry skin as in, for example, Japanese Patent Application JP 61 145 109 and European Patent Application EP 1 406 588.

The present inventors have discovered that the odor left on keratinic materials after contact with water treated with a halogenated disinfectant may be appreciably reduced by applying an effective quantity of a composition containing in a cosmetically acceptable medium at least one aminoalkane sulfonic, sulfinic or sulfonothioic acid compound or a salt thereof, an amide derivative thereof or analogs thereof on, for example, leaving the pool or other water source treated with a halogenated disinfectant.

The present disclosure thus relates to a cosmetic process to reduce or eliminate the odor retained by keratinic materials placed in contact with water treated with a halogenated disinfectant, where the process comprises applying to the keratinic materials at least one composition containing, in a cosmetically acceptable medium, at least one aminoalkane sulfonic, sulfonothioic or sulfinic acid compound, or a salt thereof, an amide derivative thereof, or a functional analog thereof, then, if necessary, rinsing with water.

The present disclosure further relates to a cosmetic composition for the purpose of reducing or eliminating the odor retained by keratinic materials placed in contact with water treated with a halogenated disinfectant, wherein said composition comprises at least one aminoalkane sulfonic or sulfonothioic acid compound or a salt thereof, or an ester or amide derivative thereof.

The various embodiments of the present disclosure are described in more detail below. All meanings and definitions provided herein with respect to the compounds used in the present disclosure are applicable to all embodiments of the present disclosure.

As used herein, “disinfectant” is understood to mean any substance capable of destroying microbial germs occurring in water that is placed into contact with human keratinic materials, for instance, skin and hair such as in, for example, the water of a swimming pool, a Jacuzzi or the water supply.

As used herein, “keratinic materials” is understood to mean the skin, the hair, the eyelashes, the eyebrows, the lips, the nails and the mucous membranes. It is also understood to mean animal keratinic materials such as hair and fur.

“Cosmetically acceptable medium,” as used herein, is understood to mean a non-toxic medium which can be applied to keratinic materials. The composition of the present disclosure may comprise a cosmetic or dermatological composition.

According to at least one embodiment, the disinfectant is chosen from chlorinated disinfectants, brominated disinfectants and mixtures thereof.

The compounds which may be used in the context of the present disclosure may be chosen, in at least one embodiment, from aminoalkane sulfonic, sulfonothionic and sulfinic acid compounds and the salts or amide derivatives or functional analogs thereof corresponding to the following formula (A) or (B):

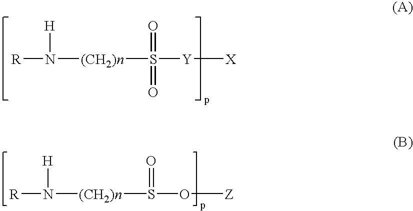

wherein

R is chosen from hydrogen and linear or branched C₁-C₈ alkyl radicals;

Y is S or O;

X is chosen from hydrogen, a cation M^{p+} of valence p, and organic amines;

Z is chosen from hydrogen, a cation M^{p+} of valence p, and organic amines;

n is an integer greater than or equal to 2; and

p is an integer greater than or equal to 1.

For example, in at least one embodiment,

R is chosen from hydrogen and linear C₁-C₄ alkyl radicals, for example, methyl;

p is 1 or 2;

n is 2 or 3;

X is chosen from hydrogen and a cation M^{p+} chosen from the alkali metals (K+, Na+), the alkaline earths (Mg²⁺, Ca²⁺) and the ammonium ion; and

Z is chosen from hydrogen and a cation M^{p+} chosen from the alkali metals (K+, Na+), the alkaline earths (Mg²⁺, Ca²⁺) and the ammonium ion.

Among the compounds of formula (A), non-limiting mention may be made, for example, of the following:

taurine or 2-aminoethanesulfonic acid;

potassium taurate, for instance, potassium taurate mixed with lauric acid (INCI name: Potassium Taurate Laurate) such as the commercial product L-TK sold by the company NOF Corporation;

sodium taurate, for example, mixed with lauric acid such as the commercial product L-T2 sold by the company NOF Corporation or mixed with N-cocoyl N-methyltaurine such as the commercial product K-02 sold by the company NOF Corporation

the sodium salt of N-methyltaurine, for instance, in the form of a mixture with N-cocoyl-N-methyltaurine (INCI name: Sodium Methyltaurine Cocoyl Methyl Taurate) such as the commercial product DIAPON K1T2 sold by the company NOF Corporation;

thiotaurine or 2-aminoethanesulfonothioic acid;

homotaurine or 2-aminopropanesulfonic acid;

Among the compounds of formula (B), non-limiting mention may be made, for example, of hypotaurine and 2-aminoethane-sulfinic acid.

As disclosed herein, the aminoalkane sulfonic, sulfonothionic or sulfinic acid compounds and the salts or derivatives thereof can be chosen from the functional analogs of taurine such as those described in the article “Taurine analogues, a new class of therapeutics: retrospect and prospects”, Gupta R C, Win T, Bittner S, Curr Med. Chem.

In at least one embodiment, such functional analogs may be chosen from taurine, homotaurine and hypotaurine in the free form, and in a further embodiment, are chosen from taurine in the free form.

According to at least one embodiment of the present disclosure, the at least one aminoalkane sulfonic, sulfonothioic or sulfinic acid compound, salt thereof, amide derivative thereof or functional analog thereof is present in the composition in an amount ranging from 0.05 to 10%, for example, ranging from 0.1 to 5% by weight, relative to the total weight of the composition.

Further, according to at least one embodiment of the present disclosure, the compositions may contain at least one surfactant agent present in an amount ranging from 0.1% to 60% by weight, for instance, ranging from 1% to 40% and even further ranging from 5% to 30%, relative to the total weight of the composition.

The at least one surfactant may be chosen from anionic, amphoteric and nonionic surfactant agents, or mixtures thereof.

Surfactants suitable for the implementation of the present disclosure may include the following non-limiting examples:

(i) Anionic Surfactants:

The nature of anionic surfactants is not critical in the context of the present disclosure and may be chosen in accordance with the general knowledge of the art.

Thus, examples of anionic surfactants useful, alone or mixed, in the context of the present disclosure include alkali metal salts, (for example, sodium, ammonium salts, amine salts, aminoalchohol salts or magnesium salts) of the following compounds: alkyl sulfates, alkyl ether sulfates, alkyl amidoether sulfates, alkylaryl polyether sulfates, monoglyceride sulfates; alkylsulfonates, alkylphosphates, alkylamide sulfonates, alkylarylsulfonates, α-olefin-sulfonates, paraffin-sulfonates; alkylsulfosuccinates, alkyl ether sulfo-succinates, alkylamidesulfosuccinates; alkylsulfosuccinamates; alkylsulfoacetates; alkyl ether phosphates; acylsarcosinates; acylisethionates and N-acyltaurates, the alkyl or acyl radical of all these different compounds comprising, in at least one embodiment, from 8 to 24 carbon atoms, and the aryl radical, in at least one embodiment, designating a phenyl or benzyl group. Non-limiting examples of other anionic surfactants that may be mentioned include, but are not limited to, fatty acids, salts such as the salts of oleic, ricinoleic, palmitic or stearic acid, copra oil and hydrogenated copra oil acids; and acyl-lactylates wherein the acyl radical contains 8 to 20 carbon atoms. Non-limiting examples of weakly anionic surfactants that may be mentioned include, but are not limited to, alkyl D galactoside uronic acids and salts thereof, and also polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkyl amido ether carboxylic acids and salts thereof, for instance, those containing from 2 to 50 ethylene oxide groups, and mixtures thereof.

In at least one embodiment, at least one anionic surfactant is chosen from the salts of alkylsulfates and alkyl ether sulfates and mixtures thereof.

(ii) Non-ionic Surfactant(s):

The non-ionic surfactants are also compounds well known per se (see, e.g., in this respect “Handbook of Surfactants” by M. R. PORTER, publ. Blackie & Son (Glasgow and London), 1991, pp 116-178) and their nature is not critical in the context of the present disclosure, thus they may be chosen in accordance with the general knowledge in the art. In at least one embodiment, the non-ionic surfactants may be chosen from, in a non-limiting manner, alcohols, alpha diols, alkylphenols and polyethoxylated, polypropoxylated or polyglycerolated fatty acids having a fatty chain containing, for example, 8 to 18 carbon atoms, wherein the number of ethylene oxide or propylene oxide groups can, for example, range from 2 to 50 and the number of glycerol groups can, for instance, range from 2 to 30. Also useful are copolymers of ethylene and propylene oxide, condensation products of ethylene and propylene oxide onto fatty alcohols; polyethoxylated fatty amides comprising from 2 to 30 moles of ethylene oxide, polyglycerolated fatty amides containing on average 1 to 5 glycerol groups, for example, 1.5 to 4; fatty acid esters of oxyethylenated sorbitan comprising from 2 to 30 moles of ethylene; fatty acid esters of sucrose, fatty acid esters of polyethylene glycol, alkylpolyglycosides, derivatives of N-alkyl glucamine, amine oxides such as the oxides of (C₁₀-C₁₄) alkylamines or oxides of N-acylaminopropylmorpholine. In at least one embodiment, alkylpolyglycosides are used herein as the non-ionic surfactants.

(iii) Amphoteric Surfactant(s):

The nature of the amphoteric surfactants is not critical in the context of the present disclosure and thus they may be chosen, in accordance with the general knowledge of the art, from, in an non-limiting manner, derivatives of aliphatic secondary or tertiary amines, wherein the aliphatic radical is a linear or branched chain containing 8 to 22 atoms of carbon and containing at least one water-solubilizing anionic group (for example carboxylate, sulphonate, sulphate, phosphate or phosphonate); the (C₈-C₂₀) alkyl betaines, sulphobetaines, (C₈-C₂₀) alkyl amido(C₁-C₆) alkyl betaines or (C₈-C₂₀) alkyl amido(C₁-C₆) alkyl sulphobetaines may also be cited.

Among the amine derivatives, non-limiting mention may be made of: the products marketed under the name MIRANOL, as described in U.S. Pat. Nos. 2,528,378 and 2,781,354 and of the structures:

R₂—CONHCH₂CH₂—N(R₃)(R₄)(CH₂COO—) (2)

wherein:

### R₂ is an alkyl radical derived from an acid R₂—COOH present in hydrolysed copra oil, or is chosen from heptyl, nonyl and undecyl radicals,

### R₃ is chosen from a beta-hydroxyethyl group and

### R₄ is a carboxymethyl group;

and

R₅—CONHCH₂CH₂—N(B)(C) (3)

wherein:

### B is —CH₂CH₂OX′,

### C is —(CH₂)_{z}—Y′, with z=1 or 2,

### X′ is the group —CH₂CH₂—COOH or a hydrogen atom,

### Y′ is —COOH or the radical —CH₂—CHOH—SO₃H, and

### R₅ is chosen from an alkyl radical of an acid R₉—COOH present in copra oil or in hydrolysed linseed oil; an alkyl radical, for example, with C₇, C₉, C₁₁ or C₁₃; a C₁₇ alkyl radical and the iso form thereof; and an unsaturated C₁₇ radical.

Such compounds are classified in the CTFA dictionary, 5^{th} edition, 1993, under the names Disodium Cocoamphodiacetate, Disodium Lauroamphodiacetate, Disodium Caprylamphodiacetate, Disodium Capryloamphodiacetate, Disodium Cocoampho-dipropionate, Disodium Lauroamphodipropionate, Disodium Caprylamphodipropionate, Disodium Capryloamphodipropionate, Lauroamphodipropionic acid and Cocoampho-dipropionic acid.

By way of example, the cocoamphodiacetate marketed under the trade name MIRANOL C2M concentrate by the company RHODIA CHIMIE may be used in at least one embodiment.

In the compositions according to the present disclosure, mixtures of surfactant agents, such as mixtures of anionic surfactant agents and mixtures of anionic surfactant agents and amphoteric or non-ionic surfactant agents are used in at least one embodiment. In at least one further embodiment, a mixture of at least one anionic surfactant and at least one amphoteric surfactant agent is used.

Non-limiting examples of at least one anionic surfactant agent may include sodium, triethanolamine and ammonium (C₁₂-C₁₄) alkyl sulfates, sodium, triethanolamine or ammonium (C₁₂-C₁₄) alkyl ether sulphates oxyethylenated comprising 2.2 moles of ethylene oxide, sodium cocoyl isethionate and sodium (C₁₄-C₁₆) alpha olefinsulphonate and mixtures thereof with:

either an amphoteric surfactant agent such as the amine derivatives named disodium cocoamphodipropionate or sodium cocoamphopropionate marketed, for example, by the company RHODIA CHIMIE under the trade name “MIRANOL C2M CONC” in aqueous solution with 38% of active substance or under the name MIRANOL C32;

or an amphoteric surfactant agent of the zwitterionic type such as the alkylbetaines, for instance, the cocobetaine marketed under the name “DEHYTON AB 30” in aqueous solution with 32% of active substance by the company COGNIS.

As disclosed herein, the compositions of the present disclosure may also contain at least one keratinic material protective agent.

The keratinic material protective agents can be any active agent useful for preventing or limiting damage due to physical or chemical attack.

Thus, the keratinic material protective agent can be chosen from water-soluble, liposoluble or water-insoluble organic UV filters, metal oxide pigments, antiradical agents, antioxidant agents, vitamins and provitamins.

The UV filters (systems filtering UV radiation) may be chosen from water-soluble and liposoluble, silicone and non-silicone, organic filters and inorganic oxide particles, the surface whereof may have been treated to render it hydrophilic or hydrophobic.

Hydrophilic polymers displaying photoprotective properties against UV radiation may also be used, for example, polymers containing benzylidene camphor and/or benzotriazole groups, substituted with sulphonate or quaternary ammonium groups.

The hydrophilic, lipophilic or insoluble organic UV filters may, in at least one-embodiment, be chosen from the anthranilates; dibenzoylmethane derivatives; cinnamate derivatives; salicylate derivatives, camphor derivatives, benzophenone derivatives, β,β-diphenyl-acrylate derivatives, triazine derivatives, benzotriazole derivatives, benzalmalonate derivatives, for instance, those cited in U.S. Pat. No. 5,624,663; benzimidazole derivatives; imidazolines; bis-benzoazolyl derivatives as described in European Patent No. 0 669 323 and U.S. Pat. No. 2,463,264; p-aminobenzoic acid (PABA) derivatives; methylene bis-(hydroxyphenyl benzotriazole) derivatives as described in U.S. Pat. Nos. 5,237,071, and 5,166,355, British Patent Application 2,303 549, German Patent Application 197 26 184 and European Patent Application 0 893 119; benzoxazole derivatives as described in European Patent Applications 0 832 642, 1 027 883, and 1 300 137 and German Patent Application 101 62 844; filter polymers and filter silicones such as those described, e.g., in International Patent Application WO-93/04665; dimers derived from α-alkylstyrene such as those described in German Patent Application 198 55 649; 4,4-diarylbutadienes as described in European Patent Applications 0 967 200, 1 008 586, 1 133 980 and 1 133 981 and German Patent Applications 197 46 654,197 55 649, and mixtures thereof.

Further examples of organic UV filters include, but are not limited to those denoted below under their INCI names:

### Para-Aminobenzoic Acid Derivatives:

### PABA,

### Ethyl PABA,

### Ethyl dihydroxypropyl PABA,

### Ethylhexyl dimethyl PABA sold, for instance, under the name “ESCALOL 507” by ISP, Glyceryl PABA, and

### PEG-25 PABA sold, for example, under the name “UVINUL P25” by BASF.

### Dibenzoylmethane Derivatives:

Butyl methoxydibenzoylmethane sold, for instance, under the trade name “PARSOL 1789” by HOFFMANN LAROCHE, and Isopropyl dibenzoylmethane.

### Salicylate Derivatives:

Homosalate sold, for example, under the name “Eusolex HMS” by Rona/EM Industries,

Ethylhexyl salicylate sold, for instance, under the name “NEO HELIOPAN OS” by Haarmann and REIMER,

### Dipropylene glycol salicylate sold, for example, under the name “DIPSAL” by SCHER, and TEA salicylate, sold, for instance, under the name “NEO HELIOPAN TS” by Haarmann and REIMER.

### Cinnamate Derivatives:

### Ethylhexyl methoxycinnamate sold, for example, under the trade name “PARSOL MCX” by HOFFMANN LA ROCHE,

### Isopropyl methoxycinnamate,

### Isoamyl methoxycinnamate sold, for instance, under the trade name “NEO HELIOPAN E 1000” by HAARMANN and REIMER,

### Cinoxate,

### DEA methoxycinnamate,

Diisopropyl methylcinnamate, and

### Glyceryl ethylhexanoate dimethoxycinnamate.

β,β-diphenylacrylate Derivatives:

### Octocrylene sold, for example, under the trade name “UVINUL N539” by BASF,

### Etocrylene, sold, for instance, under the trade name “UVINUL N35” by BASF,

### Benzophenone Derivatives:

### Benzophenone-1 sold, for example, under the trade name “UVINUL 400” by BASF,

### Benzophenone-2 sold, for instance, under the trade name “UVINUL D50” by BASF

### Benzophenone-3 or Oxybenzone, sold, for example, under the trade name “UVINUL M40” by BASF,

### Benzophenone-4 sold, for instance, under the trade name “UVINUL MS40” by BASF, Benzophenone-5,

### Benzophenone-6 sold, for example, under the trade name “Helisorb 11” by Norquay

### Benzophenone-8 sold, for instance, under the trade name “Spectra-Sorb UV-24” by American Cyanamid,

### Benzophenone-9 sold, for example, under the trade name “UVINUL DS-49” by BASF, Benzophenone-12, and

n-hexyl 2-(4-diethylamino-2-hydroxybenzoyl)-benzoate.

### Benzylidene Camphor Derivatives:

### 3-Benzylidene camphor manufactured, for instance, under the name “MEXORYL SD” by CHIMEX,

### 4-Methylbenzylidene camphor sold, for example, under the name “EUSOLEX 6300” by MERCK,

### Benzylidene camphor sulphonic acid manufactured, for instance, under the name “MEXORYL SL” by CHIMEX,

### Camphor benzalkonium methosulphate manufactured, for example, under the name “MEXORYL SO” by CHIMEX,

### Terephthalylidene dicamphor sulphonic acid manufactured, for instance, under the name “MEXORYL SX” by CHIMEX, and

### Polyacrylamidomethyl benzylidene camphor manufactured, for example, under the name “MEXORYL SW” by CHIMEX.

### Phenyl benzimidazole Derivatives:

Phenylbenzimidazole sulfonic acid sold, for instance, under the trade name “EUSOLEX 232” by MERCK, and

### Disodium phenyl dibenzimidazole tetrasulphonate sold, for example, under the trade name “NEO HELIOPAN AP” by Haarmann and REIMER.

### Phenyl benzotriazole Derivatives:

### Drometrizole trisiloxane sold, for instance, under the name “Silatrizole” by RHODIA CHIMIE, and

### Methylene bis-benzotriazolyl tetramethylbutylphenol sold, for example, in solid form under the trade name “MIXXIM BB/100” by FAIRMOUNT CHEMICAL or in micronized form in aqueous dispersion under the trade name “TINOSORB M” by CIBA SPECIALTY CHEMICALS.

### Triazine Derivatives:

### Bis-ethylhexyloxyphenol methoxyphenyl triazine sold, for instance, under the trade name <<TINOSORB S” by CIBA GEIGY,

### Ethylhexyl triazone sold, for example, under the trade name <<UVINUL T150” by BASF,

### Diethylhexyl butamido triazone sold, for instance, under the trade name “UVASORB HEB” by SIGMA 3V,

2,4,6-tris(dineopentyl 4′-amino benzalmalonate)-s-triazine, and
la 2,4,6-tris-(diisobutyl 4′-amino benzalmalonate)-s-triazine.

The symmetrical triazine filters described in International Patent Application WO 2004/085412 (see compounds 6 and 9), such as the 2,4,6-tris-(biphenyl)-1,3,5-triazines (for example, 2,4,6-tris(biphenyl-4-yl-1,3,5-triazine) and 2,4,6-tris(terphenyl)-1,3,5-triazine which is recited in the Beiersdorf Patent Applications WO 2006/035000, WO 2006/034982, WO 2006/034991, WO 2006/035007, WO 2006/034992 and WO 2006/034985.

### Anthranilate Derivatives:

### Menthyl anthranilate sold, for instance, under the trade name “NEO HELIOPAN MA” by Haarmann and REIMER.

### Imidazoline Derivatives:

### Ethylhexyl dimethoxybenzylidene dioxoimidazoline propionate.

### Benzalmalonate Derivatives

### Di-neopentyl 4′-methoxybenzalmalonate, and

### Polyorganosiloxane with benzalmalonate groups such as Polysilicone-15 sold, for example, under the trade name “PARSOL SLX” by HOFFMANN LA ROCHE.

4,4-diarylbutadiene Derivatives:

1,1-dicarboxy (2,2′-dimethyl-propyl)-4,4-diphenylbutadiene.

### Benzoxazole Derivatives:

2,4-bis-[5-1 (dimethylpropyl)benzoxazol-2-yl-(4-phenyl)-imino]-6-(2-ethylhexyl)-imino-1,3,5-triazine sold, for instance, under the name of Uvasorb K2A by Sigma 3V and mixtures thereof.

In at least one embodiment, the organic UV filters are chosen from, in a non-limiting manner:
Ethylhexyl methoxycinnamate,
Homosalate,
Ethylhexyl salicylate,
Octocrylene,
Phenylbenzimidazole sulfonic acid,
Benzophenone-3,
Benzophenone-4,
Benzophenone-5,
n-hexyl 2-(4-diethylamino-2-hydroxybenzoyl)-benzoate,
4-Methylbenzylidene camphor,
Terephthalylidene dicamphor sulfonic acid,
Disodium phenyl dibenzimidazole tetra-sulfonate,
Ethylhexyl triazone,
Bis-ethylhexyloxyphenol methoxyphenyl triazine,
Diethylhexyl butamido triazone,
2,4,6-tris(dineopentyl 4′-amino benzalmalonate)-s-triazine,
2,4,6-tris-(diisobutyl 4′-amino benzalmalonate)-s-triazine,
2,4,6-tris(biphenyl-4-yl-1,3,5-triazine),
2,4,6-tris(terphenyl)-1,3,5-triazine,
Methylene bis-benzotriazolyl tetramethylbutylphenol,
Drometrizole trisiloxane,
Polysilicone-15,
Di-neopentyl 4′-methoxybenzalmalonate,
1,1-dicarboxy (2,2′-dimethyl-propyl)-4,4-diphenylbutadiene,
2,4-bis-[5-1 (dimethylpropyl)benzoxazol-2-yl-(4-phenyl)-imino]-6-(2-ethylhexyl)-imino-1,3,5-triazine,
   and mixtures thereof.

The inorganic oxides may be chosen from the oxides of titanium, the oxides of zinc and the oxides of cerium.

The antioxidant and/or antiradical agents may be chosen from phenols such as BHA (tert-butyl-4-hydroxyanisole), BHT (2,6-di-tert-butyl-p-cresol), TBHQ (tert.-butylhydroquinone), polyphenols such as the proanthocyanidolic oligomers and the flavonoids, hindered amines known under the generic name of HALS (Hindered Amine Light Stabiliser) such as tetraminopiperidine, erythorbic acid, polyamines such as spermine, cysteines, glutathione, superoxide dismutase, and lactoferrin.

In at least one embodiment of the present disclosure, vitamins may be chosen from ascorbic acid, vitamin E, vitamin E acetate, the B vitamins, for example, vitamins B3 and B5, vitamin PP, vitamin A and derivatives thereof.

The provitamins, in at least one embodiment, may be chosen from panthenol and retinol.

According to the present disclosure, the at least one keratinic material protective agent is present in the composition in an amount ranging from 0.001% to 20% by weight, or from 0.01% to 10% by weight, for example, from 0.1% to 5% by weight relative to the total weight of the final composition.

The compositions of the disclosure may also additionally contain at least one conditioning agent.

As used herein, “conditioning agent” is understood to mean any agent having the function of improving the cosmetic properties of the skin or the hair, for example, softness, comb-out, touch, smoothing and static electricity.

The conditioning agents can be in liquid, semi-solid or solid form such as oils, waxes or gums.

The conditioning agents which may be used in the context of the present disclosure may be chosen, by way of non-limiting example, from synthetic oils such as polyolefins, plant oils, fluorinated or perfluorinated oils, natural or synthetic waxes, silicones, cationic polymers, cationic proteins and protein hydrolysates, compounds of the ceramide type, cationic surfactants, fatty amines, fatty acids and derivatives thereof, and also mixtures of these different compounds.

In at least one embodiment, the conditioning agents may be chosen from cationic polymers and silicones.

Non-limiting examples of synthetic oils include the polyolefins, for example the poly-α-olefins and even further for example:

of the polybutene type, whether or not hydrogenated, such as polybutene, whether or not hydrogenated.

In at least one embodiment, oligomers of isobutylene of molecular weight less than 1000 and mixtures thereof with polyisobutylenes of molecular weight greater than 1000, for example, ranging between 1000 and 15000, are used.

Poly-α-olefins that may be used, by way of non-limiting example, include the polyisobutenes sold, for instance, under the name of PERMETHYL 99 A, 101 A, 102 A, 104 A (n=16) and 106 A (n=38) by the company PRESPERSE Inc, or also the products sold, for example, under the name of ARLAMOL HD (n=3) by the company ICI (n indicating the degree of polymerisation),

of the polydecene type, whether or not hydrogenated.

Such products are sold, for instance, under the names ETHYLFLO by the company ETHYL CORP., and ARLAMOL PAO by the company ICI.

The animal or plant oils which may be used in at least one embodiment of the present disclosure may be chosen, by way of non-limiting example, from the group formed by sunflower, maize, soya, avocado, jojoba, gourd, grape seed, sesame, and nut oils, fish oils, glycerol tricaprocaprylate or plant or animal oils of the formula R₉COOR₁₀ wherein R₉ is the residue of a higher fatty acid containing from 7 to 29 carbon atoms and R₁₀ is chosen from a linear and branched hydrocarbon chain containing from 3 to 30 carbon atoms, such as, alkyl or alkenyl, for example, Purcellin oil or liquid jojoba wax;

Natural or synthetic essential oils such as eucalyptus, lavandin, lavender, vetiver, litsea cubeba, lemon, sandalwood, rosemary, camomile, savory, nutmeg, cinnamon, hyssop, caraway, orange, geraniol, cade and bergamot oils can also be used;

Waxes are natural (animal or plant) or synthetic substances solid at ambient temperature (20°-25° C.). They are insoluble in water, soluble in oils and are capable of forming a water-repellent film.

For the definition of waxes, reference may be made to the document, P. D. Dorgan, Drug and Cosmetic Industry, December 1983, pp. 30-33.

The wax or waxes which may be used in the context of the present disclosure may be chosen, by way of non-limiting example, from Carnauba wax, Candelila wax, and Alfa wax, paraffin wax, ozocerite, plant waxes such as olive tree wax, rice wax, hydrogenated jojoba wax or absolute waxes from flowers such as the essential wax of blackcurrant flower sold by the company BERTIN (France), animal waxes such as beeswaxes, or modified beeswaxes (cerabellina); other waxes or waxy raw materials useful according to the present disclosure are, for example, marine waxes such as those sold, for instance, by the company SOPHIM under the reference M82, polyethylene waxes or polyolefin waxes in general.

The cationic polymers which may be used in the context of the present disclosure may be chosen, by way of non-limiting example, from all those already known per se to improve the cosmetic properties of the hair, for example, those described in European Patent Application 0 337 354 and in French Patent Applications 2 270 846, 2 383 660, 2 598 611, 2 470 596 and 2 519 863.

As used herein, the expression “cationic polymer” is understood to mean any polymer containing cationic groups and/or groups ionizable to cationic groups.

Among the cationic polymers that may be used in the present disclosure, non-limiting mention may be made of those which contain primary, secondary, tertiary and/or quaternary amine groups capable either of forming part of the main polymer chain or of being borne by a side substituent directly bound to the latter.

Suitable cationic polymers may include, by way of non-limiting example, polymers of the polyamine, polyaminoamide and quaternary polyammonium type.

Polymers of the polyamine, polyaminoamide and quaternary polyammonium type useful herein may be chosen, for instance, from those described in French Patents Nos. 2 505 348 or 2 542 997. Among these polymers, the following may be cited: copolymers of acrylamide and dimethylaminoethyl methacrylate quaternized with dimethyl sulfate or with a dimethyl halide, such as that sold, for instance, under the name HERCOFLOC by the company HERCULES,

copolymers of acrylamide and methacryloyloxyethyltrimethylammonium chloride described, for example, in the European Patent Application 0 080 976 and sold, for instance, under the name BINA QUAT P 100 by the company CIBA GEIGY,

the copolymer of acrylamide and methacryloyloxyethyltrimethylammonium methosulfate sold, for example, under the name RETEN by the company HERCULES, vinylpyrrolidone/dialkylaminoalkyl acrylate or methacrylate copolymers, whether or not quaternized, such as the products sold under the name “GAFQUAT” by the company ISP, for instance, “GAFQUAT 734” or “GAFQUAT 755” or the products named “COPOLYMER 845, 958 and 937”. These polymers are described in detail in French Patents 2 077 143 and 2 393 573,

dimethyl amino ethyl methacrylate/vinylcaprolactam/vinylpyrrolidone terpolymers such as the product sold, for example, under the name GAFFIX VC 713 by the company ISP,

the vinylpyrrolidone/methacrylamidopropyl dimethylamine copolymers marketed, for instance, under the name STYLEZE CC 10 by ISP,

and vinylpyrrolidone/quaternized dimethylaminopropyl methacrylamide copolymers such as the product sold, for example, under the name “GAFQUAT HS 100” by the company ISP.

Also suitable as cationic polymers are cationic polysaccharides, for instance, celluloses in the form for example of the polymers marketed under the names “JR” (JR 400, JR 125, JR 30M) or “LR” (LR 400, LR 30M) by the company AMERCHOL and the products marketed under the name “Celquat L 200” and “Celquat H 100” by the company National Starch and the cationic galactomannan gums such as the products marketed, for example, under the trade names of JAGUAR C13 S, JAGUAR C 15, JAGUAR C 17 or JAGUAR C162 by the company RHODIA.

Also suitable for use in the present disclosure are cationic polymers chosen from:

polyaminoamide derivatives, for example, the adipic acid/dimethylamino-hydroxypropyl/diethylene triamine polymers sold, for instance, under the name “Cartaretine F, F4 or F8” by the company Sandoz,

polymers derived from polyalkylene polyamine such as those marketed, for example, under the name. “Hercosett 57” by the company Hercules Inc. or else under the name of “PD 170” or “Delsette 101” by the company Hercules in the case of the copolymer of adipic acid/epoxypropyl/diethylene-triamine,

alkyl diallyl amine or dialkyl diallyl ammonium cyclopolymers such as the polymers sold, for instance, under the name “MERQUAT 100” by the company NALCO (and homologues thereof of low weight average molecular weight) and copolymers of diallyldimethylammonium salts (for example chloride) and acrylamide, for example, marketed under the name “MERQUAT 550”,

diammonium or quaternary polyammonium polymers such as the products “Mirapol® A 15.”, “Mirapol® AD1”, “Mirapol® AZ1” and “Mirapol® 175” sold by the company Miranol,

quaternary polymers of vinylpyrrolidone and of vinylimidazole such as the products marketed, for instance, under the names Luviquat® FC 905, FC 550 and FC 370 by the company B.A.S.F,

polyamines such as Polyquart® H sold by COGNIS, referred to under the name of “POLYETHYLENEGLYCOL (15) TALLOW POLYAMINE” in the CTFA dictionary, and

polymers, for example, crosslinked, of methacryloyloxy-(C1-C4) alkyl tri(C1-C4) alkylammonium salts such as the products marketed in the form of a dispersion under the name of “SALCARE® SC 92”, “SALCARE® SC 95” and “SALCARE® SC 96” by the company CIBA.

Other cationic polymers which may be used in the present disclosure include, by way of non-limiting examples, cationic proteins and hydrolysates of cationic proteins, polyalkyleneimines, for instance, polyethyleneimines, polymers containing vinylpyridine and vinylpyridinium moieties, condensation products of polyamines and epichlorohydrin, quaternary polyureylenes and derivatives of chitin.

According to at least one embodiment of the present disclosure, the cationic polymers that are suitable for use may also include quaternary cellulose ether derivatives such as the products sold, for example, under the name “JR 400” by the company AMERCHOL, cationic cyclopolymers, such as homopolymers and copolymers of dimethyldiallylammonium salts (for example, chloride) known under the INCI name “Polyquaternium-7” such as the products sold, for instance, under the names MERQUAT 100”, “MERQUAT 550” and “MERQUAT S” by the company NALCO and homologues thereof of low weight average molecular weight, cationic polysaccharides such as guar gums modified with a 2,3-epoxypropyl trimethylammonium salt, quaternary polymers of vinylpyrrolidone and of vinylimidazole, homopolymers and copolymers, which may be crosslinked, of methacryloyloxy(C1-C4) alkyltri(C1-C4)alkylammonium salts and mixtures thereof.

The cationic polymer or polymers are present at concentrations ranging from 0.01 to 20%, for example, from 0.05 to 10% and even further for example, from 0.1 to 5% by weight relative to the total weight of the composition.

According to at least one embodiment of the compositions of the present disclosure, the silicones that are suitable for use may include volatile and non-volatile, cyclic and acyclic, branched and unbranched, organo modified and non-organo-modified silicones, such as are described below.

The silicones useful according to the present disclosure, may be soluble or insoluble in the compositions, for example, insoluble polyorganosiloxanes; they may, in at least one embodiment, take the form of oils, waxes, resins or gums.

According to the present disclosure, the silicones disclosed herein may be used as such or in the form of solutions, dispersions, emulsions, nanoemulsions or microemulsions.

The organopolysiloxanes are defined in more detail in the book by Walter NOLL “Chemistry and Technology of Silicones” (1968) Academie Press.

These silicones, as disclosed herein, can be volatile or non-volatile and may be chosen from the polyalkylsiloxanes, such as polydimethylsiloxanes with terminal trimethylsilyl groups having a viscosity of 5.10-6 at 2.5 m2/sec at 25° C., for example, 1.10-5 at 1 m2/s . The viscosity of the silicones is, for example, measured at 25° C. according to the standard ASTM 445 Appendix C.

Among these, the following commercial products may be cited in a non-restrictive manner:

the SILBIONE® oils of the 47 and 70 047 series or the MIRASIL® oils marketed by RHODIA, for example, the oil 70 047 V 500 000;

the oils of the MIRASIL® series marketed by the company RHODIA;

the oils of the 200 series from the company DOW CORNING such as DC200 of viscosity 60 000 mm2/s;

the VISCASIL® oils from GENERAL ELECTRIC and certain oils of the SF series (SF 96, SF 18) from GENERAL ELECTRIC.

Also useful herein are polydimethylsiloxanes with dimethylsilanol terminal groups, known under the name of dimethiconol (CTFA), such as the oils of the 48 series from the company RHODIA.

Non-limiting mention may also be made among this class of polyalkylsiloxanes, of the products marketed under the names “ABIL WAX® 9800 and 9801” by the company GOLDSCHMIDT which are poly(C1-C20)alkyl-siloxanes.

The polyalkylarylsiloxanes that may be used in the present disclosure may, in at least one embodiment, be chosen from the linear and branched polydimethyl/methylphenylsiloxanes and polydimethyldiphenyl-siloxanes of viscosity from 1.10-5 to 5.10-2 m2/s at 25° C.

Examples of such polyalkylarylsiloxanes include, but are not limited to:

the SILBIONE® oils of the 70 641 series from RHODIA;

the oils of the RHODORSIL® 70 633 and 763 series from RHODIA;

the oil DOW CORNING 556 COSMETIC GRAD FLUID from DOW CORNING;

the silicones of the PK series from BAYER such as the product PK20;

the silicones of the PN and PH series from BAYER such as the products PN1000 and PH1000;

certain oils of the SF series from GENERAL ELECTRIC such as SF 1023, SF 1154, SF 1250 and SF 1265.

Products useful in at least one embodiment of the present disclosure are mixtures such as:

mixtures formed from a polydimethylsiloxane hydroxylated at the end of the chain or dimethiconol (CTFA) and a cyclic poly-dimethylsiloxane also called cyclomethicone (CTFA) such as the product Q2 1401 marketed by the company DOW CORNING;

mixtures formed from a polydimethyl-siloxane gum with a cyclic silicone such as the product SF 1214 Silicone Fluid from the company GENERAL ELECTRIC, this product is an SF 30 gum corresponding to a dimethicone, having a mean numerical molecular weight of 500,000 solubilised in the oil SF 1202 Silicone Fluid corresponding to decamethylcyclopentasiloxane;

mixtures of two PDMS of different viscosities, for example, a PDMS gum and a PDMS oil, such as the product SF 1236 from the company GENERAL ELECTRIC. The product SF 1236 is the mixture of an SE 30 gum defined above having a viscosity of 20 m2/s and an SF 96 oil of viscosity 5.10-6 m2/s. This product may, for example, comprise 15% of SE 30 gum and 85% of an SF 96 oil.

Organopolysiloxane resins useful according to the present disclosure include, but are not limited to, crosslinked siloxane systems containing the moieties:

R₂SiO₂/₂, R₃SiO₁/₂, RSiO₃/₂ and SiO₄/₂ wherein R is chosen from a hydrocarbon group comprising 1 to 16 carbon atoms and a phenyl group. Among these products are those wherein R is a C₁-C₄ lower alkyl group such as methyl, or a phenyl group.

Non-limiting mention may be made among these resins, for example, of the product marketed under the name “DOW CORNING 593” or those marketed under the names “SILICONE FLUID SS 4230 and SS 4267” by the company GENERAL ELECTRIC and which are silicones of dimethyl/trimethylsiloxane structure.

Another example is resins of the trimethylsiloxysilicate type marketed, for instance, under the names X22-4914, X21-5034 and X21-5037 by the company SHIN-ETSU.

The silicones described above may be used alone or in mixtures, in a quantity ranging from 0.01 to 20% by weight, for example, ranging from 0.1 to 5% by weight relative to the total weight of the composition.

The cationic proteins or hydrolysates of proteins are, for instance, chemically modified polypeptides bearing at the end of the chain, or grafted onto the latter, quaternary ammonium groups. Their molecular weight can vary from 1500 to 10,000, for example, from 2000 to 5000. Among these compounds, the following examples may be cited:

collagen hydrolysates bearing triethylammonium groups such as the products sold, for instance, under the name “Quat-Pro E” by the company MAYBROOK and named “Triethonium Hydrolyzed Collagen Ethosulphate” in the CTFA dictionary;

collagen hydrolysates bearing trimethylammonium and trimethylstearylammonium chloride groups, sold, for example, under the name of “Quat-Pro S” by the company MAYBROOK and named “Steartrimonium Hydrolyzed Collagen” in the CTFA dictionary;

animal protein hydrolysates bearing trimethylbenzylammonium groups such as the products sold, for instance, under the name “Crotein BTA” by the company CRODA and named “Benzyltrimonium hydrolyzed animal protein” in the CTFA dictionary;

protein hydrolysates bearing on the polypeptide chain quaternary ammonium groups containing at least one alkyl radical ranging from 1 to 18 carbon atoms.

Non-limiting mention may be made among the protein hydrosylates, for example, of:
“Croquat L” the quaternary ammonium groups containing a C₁₂ alkyl group;
“Croquat M” the quaternary ammonium groups containing C₁₀-C₁₈ alkyl groups;
“Croquat S” the quaternary ammonium groups containing a C₁₈ alkyl group;
“Crotein Q” the quaternary ammonium groups containing at least one alkyl group ranging from 1 to 18 carbon atoms.

These different products are sold, for instance, by the company Croda.

Other quaternized proteins or hydrolysates are, for example, those corresponding to the formula (XIV):

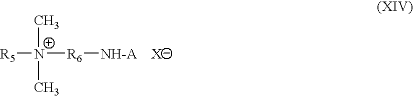

wherein X⁻ is an anion of an organic or mineral acid, A is a protein residue derived from collagen protein hydrolysates, R₅ is a lipophilic group comprising up to 30 carbon atoms, and R₆ is an alkylene group comprising 1 to 6 carbon atoms. Non-limiting mention may be made of the products sold, for example, by the company Inolex, under the name “Lexein QX 3000”, called “Cocotrimonium Collagen Hydrolysate” in the CTFA dictionary.

Non-limiting mention may be made, according to at least one embodiment of the present disclosure, of the quaternized plant proteins such as the proteins of wheat, maize and soya: as quaternized wheat proteins, those marketed, for example, by the company Croda under the names “Hydrotriticum WQ or QM”, called “Cocodimonium Hydrolysed wheat protein” in the CTFA dictionary, “Hydrotriticum QL” called “Laurdimonium hydrolysed wheat protein” in the CTFA dictionary, or again “Hydrotriticum QS”, called “Steardimonium hydrolysed wheat protein” in the CTFA dictionary.

According to at least one embodiment of the present disclosure, the compounds of the ceramide type are, for example, natural or synthetic ceramides and/or glycoceramides and/or pseudoceramides and/or neoceramides.

Non-limiting examples of compounds of the ceramide type that may be mentioned are for example described in German Patent Applications 44 24 530, 44 24 533, 44 02 929, 44 20 736, and International Applications WO 95/23807, WO 94/07844, WO 95/16665, WO 94/07844, WO 94/24097 and European Patent Applications 0 646 572, 0 227 994 and French Patent Application 2 673 179, the teachings of which are included herein by way of reference.

Compounds of the ceramide type used in at least one embodiment of the present disclosure may include:
2-N-linoleoylamino-octadecane-1,3-diol,
2-N-oleoylamino-octadecane-1,3-diol,
2-N-palmitoylamino-octadecane-1,3-diol,
2-N-stearoylamino-octadecane-1,3-diol,
2-N-behenoylamino-octadecane-1,3-diol,
2-N-[2-hydroxy-palmitoyl]-amino-octadecane-1,3-diol,
2-N-stearoyl amino-octadecane-1,3,4 triol and in particular N-stearoyl phytosphingosine,
2-N-palmitoylamino-hexadecane-1,3-diol,
(bis-(N-hydroxyethyl N-cetyl) malonamide),
the N-(2-hydroxyethyl)-N-(3-cetyloxy-2-hydroxypropyl)amide of cetylic acid, and
N-docosanoyl N-methyl-D-glucamine
   and mixtures thereof.

Cationic surfactants may also be used, included among which may be the following: salts of primary, secondary or tertiary amines, which may be polyoxyalkylenated; quaternary ammonium salts; imidazoline derivatives; or amine oxides of a cationic nature.

The quaternary ammonium salts that may be used are for example:

those which exhibit the following formula (XV):

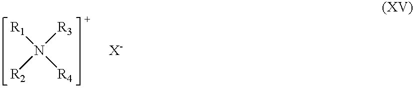

wherein the radicals R₁ to R₄, which may be the same or different, are chosen from linear or branched aliphatic radicals, comprising from 1 to 30 carbon atoms, and aromatic radicals such as aryl or alkylaryl. The aliphatic radicals may contain hetero atoms such as oxygen, nitrogen, sulphur or halogens. The aliphatic radicals are, for example, chosen from alkyl, alkoxy, polyoxy(C₂-C₆)alkylene, alkylamide, (C₁₂-C₂₂) alkyl amido (C₂-C₆)alkyl, (C₁₂-C₂₂)alkyl acetate and hydroxyalkyl radicals, comprising from 1 to 30 carbon atoms; X is an anion chosen from halides, phosphates, acetates, lactates, (C₂-C₆)alkylsulfates, alkyl- and alkylarylsulfonates,

the quaternary imidazolinium salts, such as, for example, that of the following formula (XVI):

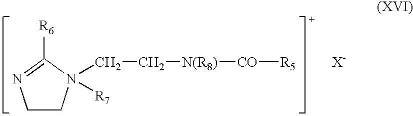

wherein R₅ is chosen from alkenyl and alkyl radicals comprising from 8 to 30 carbon atoms, for example, tallow fatty acid derivatives, R₆ is chosen from a hydrogen atom, a C₁-C₄ alkyl radical and an alkenyl or alkyl radical comprising from 8 to 30 carbon atoms, R₇ is a C₁-C₄ alkyl radical, R₈ is chosen from a hydrogen atom and a C₁-C₄ alkyl radical, and X is an anion chosen from halides, phosphates, acetates, lactates, alkylsulfates, and alkyl- or alkylarylsulfonates. For example, in at least one embodiment, R₅ and R₆ may be chosen from a mixture of alkenyl and alkyl radicals comprising from 12 to 21 carbon atoms, for example, tallow fatty acid derivatives, R₇ is methyl and R₈ is hydrogen. Such a product is marketed, for example, under the name <<REWOQUAT W 75>> by the company DEGUSSA, quaternary diammonium salts of the formula (XVII):

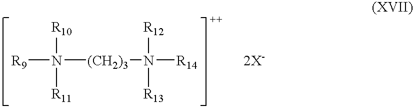

wherein R₉ is an aliphatic radical comprising 16 to 30 carbon atoms, R₁₀, R₁₁, R₁₂, R₁₃ and R₁₄, which are identical or different, are chosen from hydrogen and alkyl radicals comprising from 1 to 4 carbon atoms, and X is an anion chosen from halides, acetates, phosphates, nitrates and methylsulfates. Such quaternary diammonium salts include, for instance, propane tallow diammonium dichloride.

quaternary ammonium salts containing at least one ester function

According to at least one embodiment of the present disclosure, useful quaternary ammonium salts containing at least one ester function are, for example, those of the following formula (XVIII):

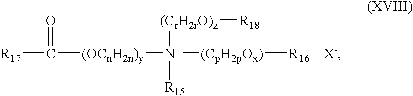

wherein:

R₁₅ is chosen from C₁-C₆ alkyl radicals, C₁-C₆ hydroxyalkyl and C₁-C₆ dihydroxyalkyl radicals;

R₁₆ is chosen from:

the

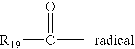

linear or branched, saturated or unsaturated C₁-C₂₂ hydrocarbon radicals R₂₀,

and a hydrogen atom,

R₁₈ is chosen from:

the

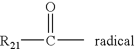

linear or branched, saturated or unsaturated C₁-C₆ hydrocarbon radicals R₂₂,

and a hydrogen atom,

R₁₇, R₁₉ and R₂₁, which may be identical or different, are chosen from linear or branched, saturated or unsaturated C₇-C₂₁ hydrocarbon radicals;

n, p and r, which may be identical or different, are integers of value ranging from 2 to 6;

y is an integer of value ranging from 1 to 10;

x and z, which may be identical or different, are integers of value ranging from 0 to 10;

X⁻ is a simple or complex, organic or inorganic anion;

provided that the sum x+y+z is from 1 to 15, that when x is 0, then R₁₆ is R₂₀ and that when z is 0 then R₁₈ is R₂₂.

The alkyl radicals R₁₁ may be linear or branched, and in at least one embodiment, linear.

In at least one embodiment, R₁₅ is chosen from methyl, ethyl, hydroxyethyl and dihydroxypropyl radicals, and in a further embodiment from methyl and ethyl radicals.

In at least one embodiment, the sum x+y+z is from 1 to 10.

When R₁₆ is a hydrocarbon radical R₂₀, it may be long and comprise from 12 to 22 carbon atoms or short and comprise from 1 to 3 carbon atoms.

When R₁₈ is a hydrocarbon radical R₂₂, it may comprise, in at least one embodiment, from 1 to 3 carbon atoms.

In at least one embodiment, R₁₇, R₁₉ and R₂₁, which may be identical or different, are chosen from linear or branched, saturated or unsaturated C₁₁-C₂₁ hydrocarbon radicals. In a further embodiment, R₁₇, R₁₉ and R₂₁, are chosen from linear or branched, saturated or unsaturated C₁₁-C₂₁ alkyl and alkenyl radicals.

In at least one embodiment, x and z, which may be identical or different, are chosen from 0 and 1, and y equals 1.

In at least one embodiment, n, p and r, which may be identical or different, are chosen from 2 and 3.

The anion is, in at least one embodiment, chosen from a halide (chloride, bromide or iodide) and an alkylsulphate, for example, methylsulfate. However, methanesulfonate, phosphate, nitrate, tosylate, an anion derived from an organic acid such as acetate or lactate or any other anion compatible with the ammonium with ester function can be used.

The anion X for example, may be chosen from chloride or methylsulfate.

In at least one embodiment, ammonium salts of formula (XVIII) may be used wherein:

R₁₅ is chosen from methyl and ethyl radicals,

x and y equal 1;

z equals 0 or 1;

n, p and r equal 2;

R₁₆ is chosen from:

the

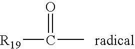

methyl, ethyl and C₁₄-C₂₂ hydrocarbon radicals,

and a hydrogen atom;

R₁₈ is chosen from:

the

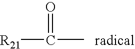

and a hydrogen atom;

R₁₇, R₁₉ and R₂₁, which may be identical or different, may be chosen from linear orbranched, saturated or unsaturated C₁₃-C₁₇ hydrocarbon radicals, such as linear or branched, saturated or unsaturated C₁₃-C₁₇ alkyl and alkenyl radicals.

According to at least one embodiment, the hydrocarbon radicals are linear.

According to the present disclosure, the compounds of formula (XVI) may be chosen from diacyloxyethyl dimethyl ammonium, diacyloxyethyl hydroxyethyl methyl ammonium, monoacyloxyethyl dihydroxyethyl methyl ammonium, triacyloxyethyl methyl ammonium, and monoacyloxyethyl hydroxyethyl dimethyl ammonium salts (e.g., chloride or methylsulfate salts) and mixtures thereof. The acyl radicals may comprise 14 to 18 carbon atoms and may be derived from a plant oil such as palm or sunflower oil. When the compound contains several acyl radicals, the latter can be identical or different.

These products are obtained, for example, by direct esterification of triethanolamine, triisopropanolamine, alkyldiethanolamine or alkyldiisopropanolamine, which may be oxyalkylenated, with fatty acids or mixtures of fatty acids of plant or animal origin or by transesterification of their methyl esters. This esterification is followed by a quaternization by means of an alkylating agent such as an alkyl (for instance, methyl or ethyl) halide, a dialkyl (for example, methyl or ethyl) sulfate, methyl methanesulfonate, methyl paratoluenesulfonate, glycol chlorohydrin or glycerol.

Such compounds are marketed, for example, under the names DEHYQUART by the company COGNIS, STEPANQUAT by the company STEPAN, NOXAMIUM by the company CECA and REWOQUAT WE 18 by the company DEGUSSA.

Ammonium salts, which may comprise at least one ester function, as described in U.S. Pat. Nos. 4,874,554 and 4,137,180 may also be used herein.

According to the present disclosure, the quaternary ammonium salts of the formula (XV) may be chosen from, by way of non-limiting examples, tetraalkylammonium chlorides such as dialkyldimethylammonium and alkyltrimethylammonium chlorides, wherein the alkyl radical comprises 12 to 22 carbon atoms, for example, behenyltrimethylammonium, distearyldimethylammonium, cetyltrimethylammonium and benzyl dimethyl stearyl ammonium chlorides and stearamidopropyldimethyl (myristyl acetate) ammonium chloride, marketed, for instance, under the name “CERAPHYL 70” by the company VAN DYK.

The fatty acids, in at least one embodiment, may be chosen from myristic acid, palmitic acid, stearic acid, behenic acid, oleic acid, linoleic acid, linolenic acid and isostearic acid.

The fatty acid derivatives may, in at least one embodiment, be chosen from esters of carboxylic acids, for example, mono, di, tri or tetracarboxylate esters.

The esters of monocarboxylic acids may, in at least one embodiment, be chosen from the monoesters of saturated or unsaturated, linear or branched C₁-C₂₆ aliphatic acids and saturated or unsaturated, linear or branched C₁-C₂₆ aliphatic alcohols, the total carbon number of the esters being greater than or equal to 10.

The monoesters, in at least one embodiment, may be chosen from dihydroabietyl behenate; octyldodecyl behenate, isocetyl behenate, cetyl lactate, C₁₂-C₁₅ alkyl lactate, isostearyl lactate, lauryl lactate, linoleyl lactate, oleyl lactate; (iso)stearyl octanoate, isocetyl octanoate, octyl octanoate, cetyl octanoate; decyl oleate; isostearyl isostearate, isocetyl laurate, isocetyl stearate, isodecyl octanoate, isodecyl oleate, isononyl isononanoate, isostearyl palmitate, methyl acetyl ricinoleate, myristyl stearate, octyl isononanoate, 2-ethylhexyl isononanoate, octyl palmitate, octyl pelargonate, octyl stearate, octyidodecyl erucate, oleyl erucate, ethyl and isopropyl palmitates, 2-ethylhexyl palmitate, 2-octyldecyl palmitate, alkyl myristates such as isopropyl, butyl, cetyl and 2-octyldodecyl myristate, hexyl stearate, butyl stearate, isobutyl stearate, dioctyl malate, hexyl laurate, 2-hexyldecyl laurate, isostearyl neopentanoate and isodecyl neopentanoate.

Esters useful herein may be chosen from C₄-C₂₂ di or tricarboxylic acids and C₁-C₂₂ alcohols and esters of mono, di or tricarboxylic acids and C₂-C₂₆ di, tri, tetra or pentahydroxy alcohols.

Non-limiting examples of esters, according to the present disclosure, that may be mentioned include: diethyl sebacate, diisopropyl sebacate, diisopropyl adipate, di-n-propyl adipate, dioctyl adipate, diisostearyl adipate, dioctyl maleate, glyceryl undecylenate, octyldodecyl stearoyl stearate; pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate and dicaprate, tridecyl erucate, triisopropyl citrate, triisostearyl citrate, glyceryl trilactate, glyceryl trioctanoate, trioctyldodecyl citrate and trioleyl citrate.

Among the esters cited above in at least one embodiment may be used: ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate, dioctyl malate, hexyl laurate, 2-hexyldecyl laurate and isononyl isononanoate, cetyl octanoate, isostearyl neopentanoate and isodecyl neopentanoate.

The fluorinated oils are, for example, the perfluoro polyethers described in European Patent Application 0 486 135 and the fluorohydrocarbon compounds described in the International Patent Application WO 93/11103, both of which are incorporated herein by way of reference.

As used herein, the term fluorohydrocarbon compounds is understood to mean compounds whose chemical structure contains a carbon skeleton wherein certain hydrogen atoms may be replaced by fluorine atoms.

The fluorinated oils can also be fluorocarbons such as fluoramines, for example, perfluorotributylamine, fluorinated hydrocarbons, for example, perfluorodecahydro-naphthalene, fluoro esters and fluoro ethers.

The perfluoro polyethers are sold, for example, under the trade names FOMBLIN by the company MONTEFLUOS and KRYTOX by the company DU PONT.

Among the fluorohydrocarbon compounds that may be mentioned are the esters of fluorinated fatty acids such as the products sold, for instance, under the name NOFABLE FO by the company NIPPON OIL.

The composition of the present disclosure may also comprise at least one mixture of conditioning agents.

Accordingly the conditioning agent or agents may be present in an amount ranging from 0.001% to 20% by weight, for example, ranging from 0.01% to 10% by weight and further for example, from 0.1 to 3% by weight relative to the total weight of the final composition.

The cosmetically acceptable aqueous medium of the compositions of the disclosure may contain, apart from water, at least one solvents chosen from the lower alcohols containing from 1 to 6 carbon atoms, such as ethanol, polyols such as glycerine, glycols such as butylene glycol, isoprene glycol, propylene glycol, dipropylene glycol, hexylene glycol (2-methyl 2,4-pentanediol), neopentyl glycol and 3-methyl-1,5-pentanediol, polyethylene glycols such as PEG-8, sorbitol, sugars such as glucose, fructose, maltose, lactose or sucrose, polyol ethers such as, for example, the monomethyl, monoethyl and monobutyl ethers of ethylene glycol or propylene glycol monomethyl ether, and alkyl ethers of diethylene glycol, such as C₁-C₄, for example, the monoethyl ether or monobutyl ether of diethylene glycol and mixtures thereof.

According to at least one embodiment of the present disclosure, at least one solvent is present in an amount ranging from 0.5 to 40% by weight, for example, ranging from 5 to 20% by weight relative to the total weight of the composition and further for example, ranging from 50 to 95% by weight of water relative to the total weight of the composition.

The compositions of the present disclosure may also comprise at least one additive normally used in the cosmetics field, for example, those used in cleansing products, perfumes, preservatives, sequestrants (EDTA), pigments, pearlescers or opacifying agents, inorganic or organic fillers, matting, whitening or exfoliant agents, soluble dyes, cosmetic or dermatological active substances, non-ionic polymers such as polyvinylpyrrolidone (PVP), anionic polymers, and fatty substances incompatible with the aqueous medium, such as oils or waxes. The quantities of these additives are those normally used in the field of art at issue, and, for example, may range from 0.01 to 20% of the total weight of the composition. These additives and the concentrations thereof must be such that they do not modify the property desired for the composition of the invention.

Non-limiting examples of a suitable oil that may be used in at least one embodiment of the present disclosure may include: oils of plant origin (jojoba, avocado, sesame, sunflower, maize, soya, safflower, grape seeds), mineral oils (vaseline, isoparaffins, which may be hydrogenated), synthetic oils (isopropyl myristate, cetearyl octanoate, polyisobutylene, ethyl-hexyl palmitate, alkyl benzoates), volatile or non-volatile silicone oils such as the polydimethylsiloxanes (PDMS) and cyclodimethylsiloxanes and cyclomethicones, and fluorinated or fluorosilicone oils, and mixtures of these oils.

Non-limiting examples of an active substance that may be used in at least one embodiment of the present disclosure may include any active substance normally used in the cosmetic and dermatological fields, for example, water-soluble or liposoluble vitamins and provitamins such as vitamins (retinol), C (ascorbic acid), B3 or PP (niacinamide), B5 (panthenol), E (tocopherol), K1 and beta-carotene, and derivatives of these vitamins such as esters thereof; steroids such as DHEA and 7α-hydroxy DHEA; antiseptics; antiseborrhoeics and antimicrobials such as benzoyl peroxide, salicylic acid, triclosan, tricarban and azelaic acid; hydrating agents such as glycerine, hyaluronic acid, pyrrolidone carboxylic acid (PCA) and salts thereof, sodium pidolate, serine, xylitol, trehalose, ectoine, ceramides and urea; keratolytic and anti-ageing agents such as the alpha-hydroxy acids such as glycolic acid, citric acid and lactic acid and beta-hydroxy acids such as salicylic acid and derivatives thereof; enzymes and co-enzymes, for example, coenzyme Q10; sun filters; optical brighteners; slenderizing agents such as caffeine, theophylline and theobromine, anti-inflammatories such as 18 β glycyrrhetinic and ursolic acids, and mixtures thereof. A mixture of two or more of these active agents may be used. The active substance or substances may be present, for instance, at a concentration ranging from 0.01 to 20%, for example, ranging from 0.1 to 10% and further for example, from 0.5 to 5% of the total weight of the composition.

Non-limiting examples of fillers that may be used in at least one embodiment of the present disclosure may include inorganic fillers such as talc and magnesium silicate (particle size: 5 microns) marketed, for example, under the name LUZENAC 15 MOO® by the company Luzenac, kaolin and aluminium silicate such as marketed, for instance, under the name KAOLIN SUPREME® by the company Imerys, and organic fillers such as starch, such as the product marketed, for example, under the name AMIDON DE MAIS B® by the company Roquette, Nylon microspheres such as those marketed, for instance, under the name ORGASOL 2002 UD NAT COS® by the company Atochem, and expanded microspheres based on vinylidene chloride/acrylonitrile/methacrylonitrile copolymer enclosing isobutane, such as those marketed, for, example, under the name EXPANCEL 551 DE® by the company Expancel. Fibers, for example, nylon fibers (POLYAMIDE 0.9 DTEX 0.3 mM marketed by Etablissements Paul Bonte) or cellulose or “rayon” fibres (RAYON FLOCK RCISE N0003 M04® marketed by the company Claremont Flock Corporation) may also be added to the composition of the present disclosure.

The compositions according to the present disclosure may be used for the washing or the treatment of keratinic materials, for example, the skin and the hair.

The compositions according to the present disclosure, may be in the form of a detergent composition such as shampoos, shower gels and foam baths. In the context of the disclosure, the compositions may contain a cleansing base, generally aqueous. The surfactant or surfactants constituting the cleansing base may be chosen, alone or as mixtures, anionic, amphoteric and non-ionic surfactants as defined above.

The type and amount of the cleansing base are chosen so as to confer on the final composition a satisfactory foaming and/or detergent power.

Thus, according to the present disclosure, the cleansing base can be present in an amount ranging from 4% to 50% by weight, for example, ranging from 6% to 35% by weight, and even further ranging from 8% to 25% by weight, of the total weight of the final composition. At least one cleansing base is present in an amount comprising at least 3% by weight of anionic surfactants, for example, from 4 to 30% by weight relative to the total weight of the composition.

The pH of the composition applied onto the keratinic materials, as disclosed herein, ranges from 2 to 11, for example from 3 to 8, and can be adjusted to the desired value by means of acidification or basification agents well known in the state of the art of compositions applied onto keratinic materials.

Non-limiting examples of the at least one basification agent may include ammonia, alkaline carbonates, alkanolamines such as mono-, di- and triethanolamines and derivatives thereof, hydroxyalkylamines and ethoxylated and/or propoxylated ethylenediamines, the hydroxides of sodium or potassium and the compounds of the following formula (XX).

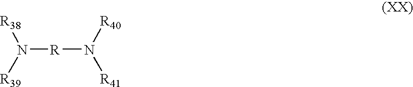

wherein R is a propylene residue, which may be substituted with a hydroxyl group or a C₁-C₄ alkyl radical, and R₃₈, R₃₉, R₄₀ and R₄₁, which may be identical or different, are chosen from a hydrogen atom and from C₁-C₄ alkyl and C₁-C₄ hydroxyalkyl radicals.

Non-limiting examples of the at least one acidification agent may include mineral and organic acids such as hydrochloric acid, orthophosphoric acid, carboxylic acids such as tartaric acid, citric acid, lactic acid and sulfonic acids.

The compositions of the present disclosure may also take the form of after-shampoos, whether or not for rinsing.

The compositions of the present disclosure may also take the form of cleansing compositions for the skin, for example, the form of solutions or gels for the bath or the shower; and the form of solid soaps.

The compositions according to the present disclosure may can also take the form of aqueous or aqueous alcoholic lotions for the care of the skin and/or the hair.

The cosmetic compositions according to the present disclosure may take the form of a gel, milk, cream, emulsion, thickened emulsion or foam.

As disclosed herein, the compositions can be packed in various forms, for example, in vaporizers, pump flasks, or in aerosol containers so as to ensure application of the composition in vaporized form or in the form of a foam. Such forms of packaging are indicated, for example, when it is desired to obtain a spray or a foam for the treatment of the skin or of the hair.

As disclosed herein, the percentages stated are by weight.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The examples that follow are intended to illustrate the present disclosure without, however, being limiting in nature.

### EXAMPLE 1

### Test of Neutralization of Odor of Water Treated with a Chlorine Disinfectant Agent

Water+commercial swimming pool chlorinating agent (ref Bayrol) at 10 g/m³, at this concentration X 10 and at this concentration X 100.

To each of these water samples contained in a vessel, an equal volume either of phosphate buffer without taurine or of phosphate buffer together with 1% by weight of taurine in the said buffer was added.

The odor was assessed blind by 6 people. Each person was asked to compare different samples containing the chlorinating agent at the same concentration with or without taurine.

The samples containing the taurine were unanimously found to release less odor than those without the taurine.

### EXAMPLE 2

### Comparative Test Between Two Shower Gels 1 and 2

The following two shower gels were prepared (the quantities are expressed in percentages by weight):

| Ingredients | Shower gel 1 | Shower gel 2 |
|---|---|---|
| (INCI NAME) | (inventive) | (comparative) |
| SODIUM CHLORIDE | 2.4 | 2.5 |
| TAURINE | 1 | 0 |
| CITRIC ACID | 0.2 | 0.2 |
| DMDM HYDANTOIN | 0.2 | 0.2 |
| SODIUM METHYLPARABEN | 0.25 | 0.25 |
| POLYQUATERNIUM-7 | 0.1 | 0.1 |
| AQUA | 72.9 | 73.8 |
| GLYCERINE | 2 | 2 |
| COCO-BETAINE | 1.5 | 1.5 |
| SODIUM LAURETH | 10.5 | 10.5 |
| SULPHATE | | |

Deodorant activity of shower gel 1 and shower gel 2 was assessed by a panel of 15 persons of European type displaying abundant or normal hairiness on the legs (calf), according to the following protocol:

the bathing was performed in the Jacuzzi with a water temperature of 40° C. and with swirling. The quantity of chlorine placed in the bath was 3 mg/l. The pH of the water after dissolution of the chlorine was 7.8.

immersion time of legs: 15 minutes

each leg was moistened (temperature 38° C. and water flow rate under the use conditions), shower gel 1 was applied in the amount of 1.2 g on the right leg (from the foot to the knee) then massaged for 1 minute. The product was left for 1 minute and then rinsed off.

wiping of each leg with a disposable towel (1 towel/leg)

pause time: 15 minutes

drying with hair dryer for 20 seconds before sniffing

direct sniffing was performed by 2 experts, using the following scale:

### Total Chlorine Intensity (T.C.I.) Scale

| 0 | 1 | 2 | 3 | 4 |
|---|---|---|---|---|
| None | Slight | Moderate | Strong | Very Strong |

The mean value of the intensity of the chlorine odor was measured and also the percentage variation in the intensity of the odor by the following calculation:

% variation=(gel 1 intensity−gel 2 intensity)X100/gel 2 intensity

A statistical test was performed on the basis of the Wilcoxon test.

### Results

| | Shower gel 1 | Shower gel 2 |
|---|---|---|
| Composition | (inventive) | (comparative) |
| Intensity of odor | 1.26 ± 0.2 | 1.75 ± 0.17 |
| % variation | 28% | — |

| | |
|---|---|
| Significance of test | yes |

The results show that the intensity of the chlorine odor was weaker on a leg treated with a shower gel 1 containing taurine compared to a leg washed with the shower gel 2 not containing taurine.

What is claimed is:

1. A cosmetic process for reducing or eliminating the odor retained by keratinic materials placed in contact with water treated with a halogenated disinfectant, comprising
applying onto the keratinic materials at least one composition comprising, in a cosmetically acceptable medium, at least one aminoalkane sulfonic or sulfonothioic acid compound, a salt thereof, an amide derivative thereof or a functional analog thereof,
then rinsing with water if necessary.

2. A process according to claim 1, wherein the halogenated disinfectant is chosen from chlorinated disinfectants and brominated disinfectants and mixtures thereof.

3. A process according to claim 1, wherein the at least one aminoalkane sulfonic, sulfonothioic or sulfinic acid compound, salt or amide derivative thereof is chosen from compounds corresponding to the following formula (A) or (B): 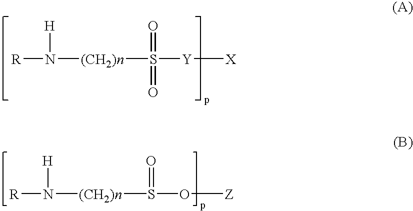
wherein
R is chosen from a hydrogen and linear or branched C₁-C₈ alkyl radicals;
Y is S or O;
X is chosen from hydrogen, a cation M^{p+} of valence p and an organic amine;
Z is chosen from hydrogen a cation M^{p+} of valence p and an organic amine;
n is an integer greater than or equal to 2; and
p is an integer greater than or equal to 1.

4. A process according to claim 3, wherein:
R is chosen from hydrogen and a linear C₁-C₄ alkyl radical
p is 1 or 2;
n is 2 or 3;
X is chosen from hydrogen and a cation M^{p+} chosen from alkali metal cations, alkaline earth metal cations, and an ammonium ion; and
Z is chosen from hydrogen and a cation M^{p+} chosen from alkali metal cations, alkaline earth metal cations, and an ammonium ion.

5. A process according to claim 4, wherein said alkali metal cations are chosen from K⁺ and Na⁺ and said alkaline earth metal cations are chosen from Mg²⁺ and Ca²⁺.

6. A process according to claim 1, wherein the at least one aminoalkane sulfonic, sulfonothioic or sulfinic acid compound, salt, amide derivative or functional analog thereof is chosen from the following compounds:
taurine or 2-aminoethanesulphonic acid;
potassium taurate;
sodium taurate;
the sodium salt of N-methyltaurine;
thiotaurine or 2-aminoethanesulfonothioic acid;
homotaurine or 2-aminopropanesulfonic acid; and
hypotaurine or 2-aminoethanesulfinic acid.

7. A process according to claim 6, wherein taurine, homotaurine and/or hypotaurine is used in the free form.

8. A process according to claim 1, wherein the at least one aminoalkane sulfonic, sulfonothioic or sulfinic acid compound, salt, amide derivative or functional derivative thereof is present in the composition in a concentration ranging from 0.05 to 10% by weight relative to the total weight of the composition.

9. A process according to claim 8, wherein the at least one aminoalkane sulfonic, sulfonothioic or sulfinic acid compound, salt, or amide derivative thereof is present in the composition in a concentration ranging from 0.1 to 5% by weight relative to the total weight of the composition.

10. A process according to claim 1, wherein the composition further comprises at least one surfactant agent.

11. A process according to claim 1, wherein the composition additionally comprises at least one keratinic material protective agent.

12. A process according to claim 1, wherein the composition additionally comprises at least one conditioning agent.

13. A process according to claim 12, wherein the at least one conditioning agent is chosen from cationic polymers and silicones.

14. A process according to claim 12, wherein the at least one conditioning agent is chosen from quaternary cellulose ether derivatives, homopolymers or copolymers of dimethyldiallylammonium salts, guar gums modified with a 2,3-epoxypropyl trimethylammonium salt, quaternary polymers of vinylpyrrolidone and of vinylimidazole, homopolymers or copolymers, which may be crosslinked, of methacryloyloxy (C1-C4) alkyltri (C1-C4) alkylammonium salts and mixtures thereof.

15. A process according to claim 1, wherein the composition additionally comprises at least one additive chosen from perfumes, preservatives, sequestrants (EDTA), pigments, pearlescers or opacifying agents, inorganic or organic fillers, matting, whitening or exfoliant agents, soluble dyes, cosmetic or dermatological active substances, non-ionic polymers, anionic polymers, and fatty substances incompatible with the aqueous medium.

16. A process according to claim 1, wherein the composition comprises a cleansing base present in an amount ranging from 4% to 50% by weight, relative to the total weight of the composition.

17. A process according to claim 16, wherein the cleansing base is present in an amount ranging from 8% to 25% by weight, relative to the total weight of the composition.

18. A process according to claim 16, wherein the cleansing base comprises at least 3% by weight of anionic surfactant, relative to the total weight of the composition.

19. A process according to claim 18, wherein the cleansing base comprises from 4 to 30% by weight of anionic surfactant, relative to the total weight of the composition.

20. A process according to claim 1, wherein the pH of the composition applied onto the keratinic materials ranges from 2 to 11.

21. A process according to claim 20, wherein the pH of the composition applied to the keratinic materials ranges from 3 to 8.
